(19) United States
(12) Patent Application Publication
(10) Pub. No.: US 2024/0280572 A1
(43) Pub. Date: Aug. 22, 2024

(54) AN ANALYTICAL DEVICE FOR QUANTITATIVE MEASUREMENT OF THE CONCENTRATION OF ANALYTES IN A LIQUID SAMPLE

(71) Applicant: BIOSTRIP APS, Risskov (DK)

(72) Inventors: Jesper Fleischer, Højbjerg (DK); Nils E. Magnusson, Lystrup (DK)

(21) Appl. No.: 18/245,938

(22) PCT Filed: Sep. 29, 2021
(86) PCT No.: PCT/EP2021/076815

§ 371 Date: Mar. 20, 2023

(30) Foreign Application Priority Data
Sep. 29, 2020 (EP) 20198945.6

(51) Int. Cl. G01N 33/543; G01N 27/12; G01N 27/22

(52) U.S. Cl. CPC G01N 33/54388; G01N 27/122; G01N 27/221; G01N 27/227; G01N 27/228

(57) ABSTRACT

An analytical device for measuring an analyte concentration in a liquid sample includes: an application zone for receiving the sample; an electrical device with: a chip and antenna for continuous bidirectional radio-wave communication with an external reader unit; a sensing circuit having a detection zone, having an immobilized capture molecule for specific interaction with the analyte, the interaction resulting in a change in a detection zone electrical property. The electrical device receives, by the chip/antenna, a radio-wave based interrogation signal from the reader unit, a current flows through the sensing circuit and is modified into a return signal representative of analyte concentration, and the return signal is radio-wave based transmitted to the reader or the chip initiates a flow of a current through the sensing circuit which is modified into a return signal representative of the analyte concentration, and the return signal is radio-wave based transmitted to the reader unit.

### CROSS-REFERENCE TO RELATED APPLICATIONS

This application is the U.S. National Stage of PCT/EP2021/076815 filed on Sep. 29, 2021, which claims priority to European Patent Application 20198945.6 filed on Sep. 29, 2020, the entire content of both are incorporated herein by reference in their entirety.

### FIELD OF THE INVENTION

The present disclosure relates to an analytical device, an analytical system, and a method for use thereof for measuring the concentration of an analyte in a liquid sample. An analytical device is configured to transmit a return signal, representative of the analyte concentration in a liquid sample, upon receiving an interrogation signal from an external reader unit.

### BACKGROUND OF THE INVENTION

Analytical devices, such as lateral flow assays are simple devices intended to detect the presence of a target substance in a liquid sample without the need for specialized and costly equipment. The assays are widely used in medical diagnostics for home testing, point of care testing or laboratory use, most well-known of lateral flow tests is the home pregnancy test which detects the presence of a certain hormone.

### SUMMARY OF THE INVENTION

The invention is disclosed herein.

In the classical sense, analytical devices, such as lateral flow assays, are used qualitatively for the purpose of detecting a specific substance, and the output will often be binary i.e., positive or negative read-out. Some handheld diagnostic devices known as lateral flow readers are used to provide a fully quantitative lateral flow assay result. Such diagnostic devices often implement optical or magnetic techniques such as image processing algorithms or magnetic field changes.

The present inventors provide herein an analytical device, capable of quantitative measurement of a target substance, and configured such that the results can be transferred via a radio-wave based signal (such as via radio-frequency identification (RFID)) to an external reader unit, for example an ordinary smartphone.

Thus, provided herein is an analytical device for quantitative measurement of the concentration of analytes in a liquid sample. The analytical device provided herein may be configured so as to convert a change in analyte concentration into a change in at least one electrical property. The analytical device may further comprise an electrical device capable of transmitting a return signal, representative of the analyte concentration, to an external reader unit. Typically, the electrical device comprises an antenna, a chip for remote communication using said antenna, and a sensing circuit capable of converting said change in analyte concentration into said change in at least one electrical property. The analytical device may be configured to transmit the return signal in response to receiving an interrogating signal from the external reader unit.

In particular, in first aspect, the present disclosure relates to an analytical device for measuring the concentration of an analyte in a liquid sample, said device comprising:
an application zone for receiving the sample;
an electrical device comprising:
a. a chip and an antenna configured for continuous bidirectional radio-wave communication with an external reader unit;
b. a sensing circuit comprising at least one detection zone, having an immobilized capture molecule configured for specific interaction with the analyte and wherein said specific interaction results in a change in at least one electrical property of said detection zone;
wherein the electrical device is configured such that,
upon receiving, by the chip and antenna, a radio-wave based interrogation signal from the external reader unit, a current flows through the sensing circuit and is modified into a return signal representative of the analyte concentration, said return signal is radio-wave based transmitted to the external reader or
the chip initiates a flow of a current through the sensing circuit which current is modified into a return signal representative of the analyte concentration, said return signal is radio-wave based transmitted to the external reader unit.

In specific examples, the one or more detection zones are separated from the application zone. In such examples, it is a preference that the analytical device comprises a porous phase for transporting at least part of the sample from the application zone to the one or more detection zone, as such the analytical device may comprise or consist of a lateral flow assay. However in other examples, the application zone comprises the one or more detection zones. For example, the sensing circuit may overlap, partially or completely, with the application zone. As such, the chip and antenna may also overlap with the application zone, although in specific examples they are provided separate from the application zone.

It is a preference that the immobilized capture molecule is configured such that the specific interaction with the analyte results in a direct and/or indirect change in the at least one electrical property of the detection zone. For example a binding molecule and/or a capture molecule may comprise a metal reporter that accumulates at the detection zone at a level that corresponds to the analyte concentration. The metal report may lead to a change in at least one electrical property of the sensing circuit, such that, when a current flows in said circuit, a return signal is generated. It is a preference that the sensing circuit comprises one or more detection zones and one or more reference zones, wherein the reference zones is an area of the sensing circuit that is arranged to be in contact with the liquid sample during use, however said reference zones do preferably not comprise capture molecules. As such, the reference zones may provide a reference value, for deriving a true concentration value for the one or more analytes.

In an embodiment of the present disclosure, the electrical device is configured for being powered by the wireless interrogating signal, an internal power source, such as a battery, or a combination thereof. The internal power source may be any type of power source including power sources relying on external factors such as solar, wind or electromagnetic waves.

In an embodiment of the present disclosure, the analytical device comprises an electronic device that is configured to be powered by the interrogating signal from the external reader unit. Thereby, the electronic device may be a passive device, configured for passive radio-wave based communication. Such a configuration of the analytical device may be configured to be operated without a battery, instead the only power source for operation of the analytical device may be the interrogating signal. Consequently, the electronic device may comprise or consist of a passive device and/or a passive tag.

However, in an alternative embodiment of the present disclosure, the analytical device comprises a power source for at least partly powering the electrical device. The power source may be a battery, such as a printed battery. Consequently, the electronic device may be an active device or an active tag. In this configuration of the analytical device, the electrical device may be configured to transmit data, such as from a sensing circuit, continuously and uninterruptedly, regardless of whether it is in the field of action of an external reader unit.

Any suitable network standard may be used for the communication between the analytical device and the external reader unit, for example RFID, NFC, Wi-Fi, ultra-wide band, Bluetooth, and/or a ZigBee. The electronic device may therefore comprise or consist of an RFID tag, a Wi-Fi tag, an ultra-wide band tag, a Bluetooth tag, and/or a ZigBee tag.

It should be noted that the presently disclosed analytical device may be configured in multiple ways for measuring the analyte concentration. For example, the electrical device typically comprises an antenna, a chip configured for radio-wave based communication with an external reader unit using the antenna, and a sensing circuit. However, while the antenna may be a separate unit, it could also be provided as a part of the chip, the antenna may for example be an integral part of the chip. Similarly, the antenna could be provided at least a part of the sensing circuit. Such a configuration could be made possible by having an antenna that is disposed at the porous phase, and wherein at least a part of said antenna that is disposed at the porous phase comprises capture molecules. Consequently, several different configurations of the analytical device and the electrical device are possible, and within the scope of the present disclosure.

In a second aspect, the present disclosure relates to an analytical system for measuring the concentration of at least one analyte in a liquid sample, said system comprising:
a. the analytical device as described herein; and
b. an external reader unit.

The external reader unit may be configured for generating an interrogating signal for powering the electrical device of the analytical device, and/or the external reader unit may be configured for receiving and/or interpreting a radio-wave based return signal from the electrical device, said return signal representative of the analyte concentration and/or changes in the electrical property of the at least one detection zone.

In a third aspect, the present disclosure relates to a method for measuring the concentration of an analyte in a liquid sample, said method comprising the steps of:
providing an analytical system, preferably as defined herein;
applying a liquid sample comprising analytes to an application zone of said analytical system;
generating, by the electrical device, a radio-wave based return signal
representative of the analyte concentration;
receiving, by the external reader unit, the return signal; and
optionally, interpreting said return signal;
a. thereby measuring the concentration of the analyte in the liquid sample.

In an embodiment of the present disclosure the method comprises allowing for migration of the analytes from the application zone to the at least one detection zone.

In an embodiment of the present disclosure the method generating, by an external reader unit, a radio-wave based interrogating signal.

In an embodiment of the present disclosure the method comprises receiving, by the electrical device, the interrogating signal, such that a current flows through the sensing circuit; and

### BRIEF DESCRIPTION OF DRAWINGS

FIG. 1 shows a schematic illustration of an analytical device and an external reader unit according to a specific embodiment of the present disclosure;

FIG. 2 shows a schematic illustration of an analytical device transmitting a return signal according to a specific embodiment of the present disclosure;

FIG. 3 shows a schematic illustration of an analytical device comprising binding molecules and capture molecules according to a specific embodiment of the present disclosure;

FIG. 4 shows a schematic illustration of an analytical device comprising an electrical device according to a specific embodiment of the present disclosure;

FIG. 5 shows schematic illustrations of analytical devices comprising electrical devices according to specific embodiments of the present disclosure; and

FIG. 6 shows schematic illustrations of modes of powering analytical devices comprising electrical devices according to specific embodiments of the present disclosure.

### DETAILED DESCRIPTION OF THE INVENTION

As used herein, the singular forms “a”, “an” and “the” include plural referents unless the context clearly states otherwise. Thus, for example, reference to “an antibody” includes a plurality of such antibodies.

As used herein, the term “analytical device”, refers to a device configured for quantitatively or qualitatively assessing the presence of an analyte in a liquid solution. Commonly an analytical device comprises a sensor, for example a biosensor, for assessing the presence of the analyte.

As used herein, the term “lateral flow device”, refers to an analytical device comprising a porous phase for wicking an analyte from an application zone to at least one detection zone, wherein the detection zone comprises molecules that interact with the analyte and causes a measurable signal of the presence of the analyte. Although lateral flow devices are typically provided in the form of an elongated strip, the present disclosure is not limited to a specific shape of form. Thereby, in addition to having a substantially elongated shape, the lateral flow devices of the present disclosure may be provided in any shape, for example a circular, an oval, a quadratic, or an irregular shape.

As used herein, the term “analyte” refers to any component, substance or chemical or biochemical constituent that is of interest. Within the scope of invention are molecules, including macromolecules, comprised in liquid samples, including biological samples, such as bodily samples, such as antigens, proteins, enzymes, peptides, polysaccharides, oligosaccharides, hormones, including growth hormones. Of particular interest are analytes that are biological markers for a disease or a medical or other condition. Biological markers can be of various natures and sizes.

As used herein, the expression “radio frequency identification” or “RFID” means a system comprising a radio frequency identification tag made up of a chip (microchip) with an antenna, and a radio frequency identification interrogator or radio frequency identification reader with an antenna. The radio frequency identification reader sends out electromagnetic waves. The tag antenna is tuned to receive these waves. A passive radio frequency identification tag draws power from the field created by the reader and uses it to power the circuits of the microchip. The microchip then modulates the waves that the passive radio frequency identification tag sends back to the radio frequency identification reader, which converts the waves received by the radio frequency identification reader into digital data.

As used herein “antibody” refers to antibody or antibody fragment, or a combination of antibody and fragments, which can be a monoclonal antibody, a polyclonal antibody, a chimeric antibody, or a humanized antibody. The antibody fragment of the invention comprises any fragment size, such as large and small molecules of, for example, the antibody which retain the characteristic to recognize and bind the target antigen as the antibody.

The term “aptamer” as used herein refers to a single-stranded oligonucleotide (single-stranded DNA or RNA molecule) that can bind specifically to its target with high affinity. The aptamer can be used as a biosensor element capable of binding to a molecule in a detection/analysis system, and thus has been recognized as a substitutive for antibody. Particularly, the aptamers can be used as molecules targeting various organic and inorganic materials, including toxins, unlike antibodies, and once an aptamer binding specifically to a certain material is isolated, it can be consistently reproduced at low costs using automated oligomer synthesis methods. Further, the term “affimer” refers to small, engineered non-antibody binding proteins that can bind target molecules and are designed to mimic the molecular recognition characteristics of monoclonal antibodies. Affimer reagents are suitable for use in for example biosensors and point-of-care diagnostics.

The term “affimer” refers to small, engineered non-antibody binding proteins that can bind target molecules and are designed to mimic the molecular recognition characteristics of monoclonal antibodies. Affimer reagents are suitable for use in for example biosensors and point-of-care diagnostics.

### Application Zone

The term application zone refers to the part of the device to which a sample is applied. The application zone is configured such that it allows application of a liquid sample, such as, but not limited to, a sample of blood, urine or interstitial fluid. In a preferred embodiment of the present invention, the application zone consists of part of a strip consisting of blotting paper and a membrane. In another embodiment, the application zone consists of a recess. The application zone may contain a binding molecule which is not immobilized and which is capable of binding specifically the analyte of interest. Application of the sample to the application zone allows binding of this specific binding molecule to the analyte. The application zone can contain several specific molecules, each capable of binding a different analyte.

### Biological Markers

By biological marker is understood any analyte which is characteristic of a biological condition. Of particular interest are biological markers that characterize a disease or a medical condition, as defined above. Biological markers can be of various natures and sizes. Any biological marker which can be bound specifically either by at least two different molecules or by one molecule capable of binding the biological marker in at least two different sites is within the scope of the invention. For example, a protein which can be bound by two different proteins is within the scope of the invention. Another example is a protein which can be bound simultaneously by a single other protein in two distinct sites. Another example is a protein which exists in a multimeric form, such as dimeric form, in which one monomer can be specifically bound by one protein such as an antibody, and the other monomer(s) can be specifically bound by another protein such as an antibody.

The C-reactive protein (CRP), the tumor factor p53 and the rheumatoid factor (RF) are biological markers of particular interest for the present invention. Particular embodiments of the invention include but are not limited to the following biological marker/specifically-binding molecule conjugates: protein/protein, enzyme/substrate, antigen/antibody, protein/vitamin.

### Bodily Samples

The term bodily samples is used herein in the broadest sense of the term as referring to samples taken from an organism. This organism may be the body of an animal, e.g. humans, farm animals, fish, or the organism may be a plant.

### Conjugate Pad

The conjugate pad disclosed herein is made in a suitable material capable of storing the binding molecules. Preferably, the binding molecules are stored in a dry format, and when contacted by the sample and/or the running buffer starts to migrate towards the distal end of the analytical device. Typically the conjugate pad is provided in glass fiber, but other materials are known to a person skilled in the art.

### Colloidal Metal

Colloidal metal is a suspension (or colloid) of sub-micrometer-sized particles of metal in a fluid, such as, but not limited to, water. Any colloidal metal can be used in the present invention. Colloidal metal includes any water-insoluble metal particle or metallic compound dispersed in liquid water, a hydrosol or a metal sol. Preferred metals include gold, silver and platinum. The colloidal metal may, in a preferred embodiment of the present disclosure, act as a metal reporter, wherein the amount of the metal reporter at the detection zone is representative of the analyte concentration in the sample.

### Conjugation

Conjugation is the process by which the colloidal metal particles are bound (conjugated) to a molecule such as a protein. One embodiment of the present invention relates to a protein, in particular an antibody, conjugated to colloidal metal particles, where the colloidal metal is a noble metal, in particular gold, silver and platinum. Conjugation can be performed by adsorption of the metal particles to the protein or antibody, or by covalent binding of the metal particles to thiol groups presented by the protein or antibody.

### Detection Zone

The term detection zone refers to the part of the device in which concentration of an analyte is determined by measuring a change in at least one electrical property, such as the impedance, of the said detection zone. In a preferred embodiment of the present invention, the detection zone consists of part of a strip consisting of blotting paper and a membrane. The detection zone contains a molecule which is immobilized and which is capable of binding specifically the analyte of interest. Migration of the analytes contained in the sample and applied to the application zone through the detection zone allows binding of this specific molecule to the analyte. The analyte specifically bound by the immobilized molecule conjugated to the metal reporter is thus retained in the detection zone. The detection zone can be subdivided in several detection zones, each containing a specific immobilized molecule which can be different from or identical to the immobilized molecules in any other detection zone. In one embodiment, at least one of the detection zones contains immobilized bovine serum albumin (BSA). In another embodiment, at least one of the detection zones contains a positive control. The detection zone is such that impedance can be measured with electrodes. The impedance measured in the detection zone varies as a function of the amount of metal particles retained in the said detection zone.

### Electrical Impedance

Electrical impedance is the measure of the resistance that a circuit presents to the passage of a current when a voltage is applied. In quantitative terms, it is the complex ratio of the voltage to the current in an alternating current (AC) circuit. Impedance extends the concept of resistance to AC circuits, and possesses both magnitude and phase, unlike resistance, which has only magnitude. In direct current (DC) circuits, there is no distinction between impedance and resistance; the latter can be thought of as impedance with zero phase angle. It is necessary to introduce the concept of impedance in AC circuits because there are other mechanisms impeding the flow of current besides the normal resistance of DC circuits. There are an additional two impeding mechanisms to be taken into account in AC circuits: the induction of voltages in conductors self-induced by the magnetic fields of currents (inductance), and the electrostatic storage of charge induced by voltages between conductors (capacitance). The impedance caused by these two effects is collectively referred to as reactance and forms the imaginary part of complex impedance whereas resistance forms the real part. Measurement of impedance requires measurement of the magnitude of voltage and current, and the phase difference between them. The symbol for impedance is Z and impedance is expressed in ohms (Ω).

### Electrodes

An electrode is an electrical conductor used to make contact with a non-metallic part of a circuit. Electrodes are herein used to make contact between the reader unit and the detection zones. In one embodiment, the impedance matching of the electrodes is achieved by coating the electrodes with a salt such as silver chloride. Embodiments of the present invention relate to devices in which the impedance of the detection zones is measured with at least two electrodes. In one embodiment, two electrodes are used for measuring impedance in at least two detection zones; the electrodes are moved relative to the device so that they can be contacted sequentially with each detection zone. In another embodiment, two electrodes are used for measuring impedance in each detection zone; in this embodiment, the number of electrodes is the double of the number of detection zones, and the impedances of each detection zone can be measured simultaneously. In a preferred embodiment, the impedance of each detection zone is measured with three electrodes. In another preferred embodiment, the impedance of each detection zone is measured with four electrodes.

### Liquid Sample

The term liquid sample is to be understood as any sample in the liquid form, containing analytes. The liquid sample may for example be a bodily sample, such as a urine sample, a blood sample, an interstitial fluid sample, or a plant sample. The bodily sample may originate from a mammal, such as a human or an animal (i.e. a veterinary sample). Alternatively, the liquid sample may be a non-bodily sample, such as an environment sample. The environment sample may be a sample obtained from any environment, such as a waste water sample, a sewage sample, a groundwater sample, a lake water sample, and/or a sea water sample. The volume of the sample is such that migration of the analytes contained in the sample from the application zone to the detection zone is possible; in particular the analytes shall be capable of migrating at least until beyond the last detection zone.

### Membrane

A membrane in the context of the present invention is a carrier such as a nitrocellulose, a polyvinylidene fluoride (PVDF) or a nylon membrane, or any membrane known in the art, to which molecules such as antibodies can be immobilized.

### Metal

The term “metal” as used herein is an element, compound, or alloy that is a good conductor of both electricity and heat, which readily loses electrons to form positive ions (cations), and that belongs to the “metal” group as known in the state of the art and as defined by its position on the periodic table. Of particular relevance for the present invention are metal particles which can be conjugated to antibodies.

As used herein “polymer” refers to macromolecular materials having at least five repeating monomeric units, which may or may not be the same. The term “polymer”, as used herein, encompasses homopolymers and copolymers. As used herein, the term “metal colloid” refers to a colloid in which the suspended microscopic nanoparticles are metal nanoparticles.

The terms “near-field communication” or “NFC” as used herein, refer to a set of communication protocols for communication between two electronic devices over a distance of 4 cm (1½ in) or less.

### Sensing Circuit

The term “sensing circuit” as used herein refers to an electrical circuit that is configured to generate a return signal representative of the presence of the analyte in the liquid solution. The sensing circuit comprises at least one detection zone, wherein the detection zone is at least partly exposed to the analyte or sample during use of the analytical device. The detection zone may for example overlap, partially or completely, with the application zone. The sensing circuit may be an electrical circuit that is separate, while still electrically connected, to the chip. In such instances, the sensing circuit may comprise a sensing unit for controlling the sensing circuit. The sensing circuit may further be controlled by the chip itself. The sensing circuit may further comprise an antenna. In such an instance, the electrical device, comprising the sensing circuit, is preferably configured to transmit the return signal without the signal first being provided to the chip.

A chip as used herein refers to a set of electronic circuits on one small flat piece of semiconductor material, for example silicon. Chip is used interchangeably with integrated circuits, IC and microchip.

An antenna as used herein is defined as the interface between radio waves propagating through space and electric currents moving in metal conductors. In transmission, a radio transmitter supplies an electric current to the antenna, such as the antenna's terminals, and the antenna radiates the energy from the current as electromagnetic waves (radio waves). In reception, an antenna intercepts some of the power of a radio wave in order to produce an electric current, such as at its terminals.

A reader unit is any device capable of receiving a return signal, transmitted by the electrical device. The reader unit is adapted to communicate with the analytical device while not in direct physical contact with said analytical device. Preferably the reader unit comprises a display unit, which displays the impedance of the detection zone. More preferably, the reader unit is adapted to display the concentration of the analyte of interest, for example converted from an impedance value measured in a detection zone. The reader unit may be connected to a power source or a printer unit. In a preferred embodiment the reader unit is powered with batteries and is portable. The reader unit may comprise a data storage unit.

### DETAILED DESCRIPTION

The present disclosure relates to analytical devices for assessing a concentration of at least one analyte in a liquid sample.

In one embodiment of the present disclosure, the analytical device is configured for measuring the concentration of an analyte in a liquid sample, said device comprising:
an application zone for receiving the sample;
an electrical device comprising:
a. a chip and an antenna configured for continuous bidirectional radio-wave communication with an external reader unit;
b. a sensing circuit comprising at least one detection zone, having an immobilized capture molecule configured for specific interaction with the analyte and wherein said specific interaction results in a change in at least one electrical property of said detection zone;
wherein the electrical device is configured such that,
upon receiving, by the chip and antenna, a radio-wave based interrogation signal from the external reader unit, a current flows through the sensing circuit and is modified into a return signal representative of the analyte concentration, and said return signal is radio-wave based transmitted to the external reader; or
the chip initiates a flow of a current through the sensing circuit which current is modified into a return signal representative of the analyte concentration, and said return signal is radio-wave based transmitted to the external reader unit.

### Sensing Configuration

A sensing circuit is preferably arranged to generate a return signal, representative of the analyte concentration. The sensing circuit may be arranged to sense output voltage, and provide a signal that is proportional to the output voltage, thereby returning a signal that is representative to the analyte concentration. The sensing circuit may be configured in different ways.

Thus, in one embodiment of the present disclosure, the sensing circuit of said analytical device is provided as a circuit separate from the antenna and the chip, and wherein the sensing circuit is in electrical contact with the antenna and/or the chip. The chip and antenna is preferably configured for radio-wave based communication with an external reader unit, and configured to receive an interrogation signal by an external reader unit. Preferably the interrogation signal powers components of the electrical device.

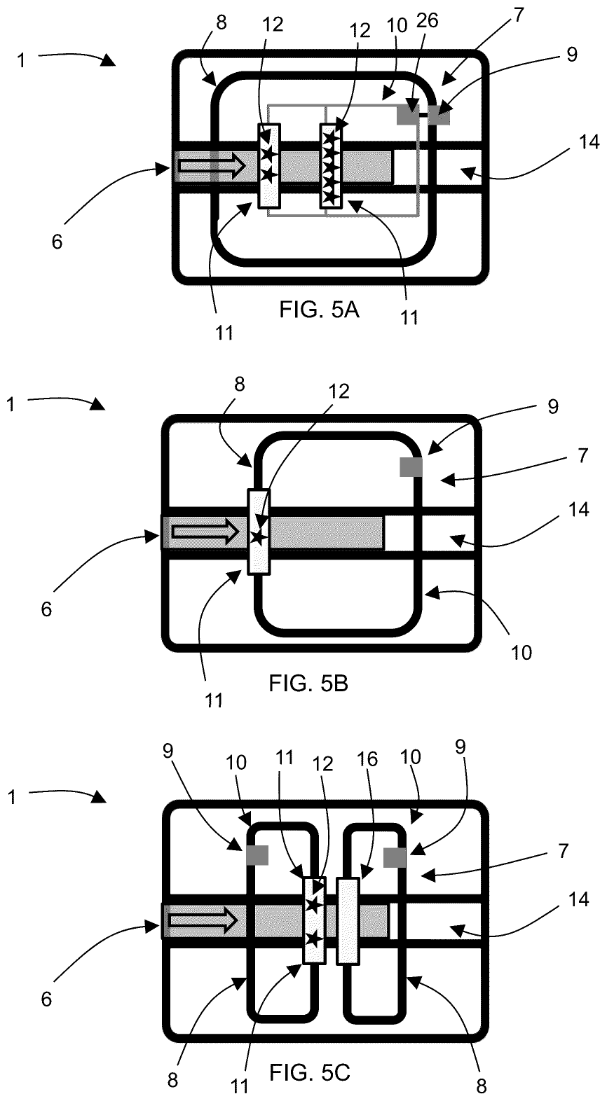

For example, the interrogation signal may provide power for operation of the one or more chips and/or the one or more sensing circuits. Further, the one or more chips and/or the one or more sensing circuits may be configured such that upon receiving the (radio-wave based) interrogation signal from an external reader unit, a current flows through the one or more sensing circuit and is modified into one or more a return signals representative of the analyte concentrations. Thus, the electrical device may comprise multiple sensing circuits, and where each of said multiple sensing circuit may be arranged such that a current flows in said each sensing circuit, upon receiving an interrogation signal by the electrical device. While multiple return signals may be generated, it is typically a preference that a single return signal is generated. This may be accomplished by the use of a chip or a sensing unit (as illustrated in FIG. 5A) that is configured to generate a return signal, based on the flow of current in each sensing circuit, as for example illustrated in FIG. 5A. The sensing unit is preferably arranged to provide processed or unprocessed measurement data to the chip. The chip and/or antenna is configured to thereafter transmit the return signal to an external reader unit. As such, the return signal may be a coded signal that comprises information of the concentration of different types of analytes in the sample.

Similarly, the analytical device may comprise one or more reference zones, typically one reference zone per detection zone. The sensing unit and/or chip may be configured similarly to as described above for cases where the analytical device comprises multiple sensing circuits, i.e. the sensing unit and/or chip may be configured such that, upon receiving, by the chip and antenna, a radio-wave based interrogation signal from the external reader unit, a current flows through each of said detection zones and reference zones. In such a case, a sensing unit and/or chip may be arranged to process the return signal to comprise measurement data of both the analyte concentration and the reference values. Preferably the return signal comprises processed measurement data comprising data of analyte concentrations that have been calculated based on the reference values, i.e. the reference values may have been used to calibrate the signals obtained from the detection zones.

In specific examples of the present disclosure, the electrical device is configured such that, upon receiving the interrogation signal, the chip activates a sensing circuit. Said activation of the sensing circuit may be powered by the interrogation signal or, fully or partly, by a battery. Preferably said battery forms part of the electrical device, the battery may for example comprise one or more thin-film batteries.

In one embodiment of the present disclosure, said sensing circuit is provided as at least a part of the chip.

In one embodiment of the present disclosure, said sensing circuit is provided as at least a part of the antenna. The antenna may for example comprise one or more type of immobilized capture molecules, configured for specific interaction with the analyte and/or the binding molecule such that said interaction results in a change in at least one electrical property of the at least one detection zone by modifying the amount of a metal reporter of said detection zone. In specific embodiments of the present disclosure, the electrical device may comprise multiple antennas, and the multiple sensing circuits, that are provided as at least a part of each antenna. Multiple sensing circuits may allow for

### Sensing Circuit Components

The sensing circuit of the analytical device may be configured in a variety of ways to accommodate for various needs, such as the need for targeting different or multiple analytes in a liquid sample, such as a biological sample.

Thus, in one embodiment of the present disclosure, the sensing circuit of said analytical device comprises at least two detection zones, such as for measuring multiple analyte concentrations. The detection zone is the part of the device in which concentration of an analyte is determined by measuring the impedance of the said detection zone.

In one embodiment of the present disclosure, said sensing circuit further comprises a reference zone configured for measuring a baseline of a measured electrical property, such as being located towards the distal end of said porous phase and wherein said reference zone does not comprise immobilized capture molecules.

In one embodiment of the present disclosure, the at least one detection zone and/or reference zone of said analytical device is a conductive film layer covering a part of the porous phase. The conductive film layer may have any shape, for example it may be a substantially flat metal film, e.g. a high aspect ratio structure, or have the shape of a wire, e.g. with a substantially circular cross section. The conductive film layer may be provided in a conductive material, for example a metal. The conductive film layer may be a part of the antenna and/or the sensing circuit.

### Electronic Signal and Generation Thereof

In order for the analyte concentration to be transferred to an external reader unit, such as a smartphone, an electronic signal needs to be generated and processed.

In one embodiment of the present disclosure, the at least one electrical property of said analytical device is selected among the group of: electrical impedance, such as the complex impedance, electrical resistance, electrical conductance, dielectric constant, frequency tuning, activation power, received signal strength indicator, emitted return signal strength or a combination thereof.

The activation power is typically the power required for activation of the device, and is generally considered as the power received by the analytical device required to activate the device, e.g. the power of the interrogation signal received by the antenna. The power of the interrogation signal (as received by the antenna) may depend on factors such as the emitted interrogation signal strength, the line of sight, and the distance between the external reader unit and the analytical device. If factors such as said distance and line of sight is constant, the power of a received interrogation signal required to activate the device may depend solely, or at least to a significant degree, on the change in at least one electrical property of the at least one detection zone, due to interaction between capture molecules configured for specific interaction with the analyte and/or the binding molecule.

The received signal strength indicator (RSSI) is the relative received signal strength in a wireless environment, in arbitrary units. It may for example be the signal strength of the return signal, as received by the external reader, or the emitted signal strength (return signal) of the device.

Electrical impedance is the measure of the resistance that a circuit presents to the passage of a current when a voltage is applied. In quantitative terms, it is the complex ratio of the voltage to the current in an alternating current (AC) circuit.

In one embodiment of the present disclosure, the antenna of said analytical device is configured for generating a radio-wave based return signal, said return signal representative of the measured electrical property and/or the analyte concentration.

In one embodiment of the present disclosure, the electrical device of said analytical device comprises a processing unit configured for processing data associated with the measurement, such as for computing the analyte concentration.

In one embodiment of the present disclosure, the interrogating signal of said analytical device is configured for powering the electrical device.

In one embodiment of the present disclosure, the electrical device of said analytical device is configured for being powered solely by the interrogating signal.

In one embodiment of the present disclosure, said electrical device consists or comprises an RFID tag, an NFC tag, a Wi-Fi tag, an ultra-wide band tag, a Bluetooth tag, and/or a ZigBee tag.

Near-Field Communication (NFC) is a set of communication protocols for communication between two electronic devices over a distance of up to 4 cm. NFC is based on inductive coupling between two antennas, communicating in one or both directions, using a frequency of or around 13.56 MHz, typically using the ISO/IEC 18000-3 air interface standard at data rates ranging from 106 to 424 Kbit/s.

Wi-Fi is a family of wireless network protocols, based on the IEEE 802.11 family of standards, commonly using the 2.4 gigahertz (120 mm) UHF radio band and/or the 5 gigahertz (60 mm) SHF radio band.

Ultra-wideband (UWB) refers to radio technology with a bandwidth exceeding the lesser of 500 MHz or 20% of the arithmetic center frequency, according to the U.S. Federal Communications Commission (FCC). UWB transmissions transmit information by generating radio energy at specific time intervals and occupying a large bandwidth, thus enabling pulse-position or time modulation. The information can also be modulated on UWB signals (pulses) by encoding the polarity of the pulse, its amplitude and/or by using orthogonal pulses.

Bluetooth is a short-range wireless technology standard that is used for exchanging data between fixed and mobile devices over short distances using UHF radio waves in the ISM bands, from 2.402 GHz to 2.48 GHz

ZigBee operates in the industrial, scientific and medical (ISM) radio bands: 2.4 GHz in most jurisdictions worldwide; though some devices also use 784 MHz in China, 868 MHz in Europe and 915 MHz in the US and Australia, however even those regions and countries still use 2.4 GHz for most commercial ZigBee devices for home use. Data rates vary from 20 Kbit/s (868 MHz band) to 250 Kbit/s (2.4 GHz band).

ZigBee builds on the physical layer and media access control defined in IEEE standard 802.15.4 for low-rate wireless personal area networks (WPANs). The specification includes four additional key components: network layer, application layer, ZigBee Device Objects (ZDOs) and manufacturer-defined application objects. ZDOs are responsible for some tasks, including keeping track of device roles, managing requests to join a network, as well as device discovery and security.

It is a preference that the communication between the analytical device and the external reader unit is bidirectional, such as by near field communication, Bluetooth, Bluetooth low energy, or Wi-Fi. Further, it is a strong presence that the communication allows for continuous sampling, such as by near field communication, Bluetooth, Bluetooth low energy, or Wi-Fi. This allows for continuous assessment of the concentration of the analyte.

In one embodiment of the present disclosure, the antenna of said analytical device is omnidirectional or hemispherical such that it is configured to receive the interrogating signal when the external reader unit is out of sight.

In one embodiment of the present disclosure, the electrical device is adapted to generate a single return signal representative of the concentrations of the at least two analytes. As such, the external reader unit, may be arranged to interpret the return signal and derive the concentration values for the at least two analytes.

In one embodiment of the present disclosure, the sensing circuit comprises a reference zone, such as one reference zone for each detection zone, wherein the one or more reference zones are configured for measuring a reference value, such as a baseline, of a measured electrical property and/or an analyte concentration.

In one embodiment of the present disclosure, the analytical device comprises one application zone for each detection zone and reference zone.

In one embodiment of the present disclosure, the one or more reference zones comprises a conductive film layer without immobilized capture molecules.

In one embodiment of the present disclosure, the at least one detection zone and/or reference zone comprises a conductive film layer covering a part of the porous phase.

In one embodiment of the present disclosure, the antenna and the chip are not part of the sensing circuit, and wherein the sensing circuit is in electrical contact with the antenna and/or the chip.

In one embodiment of the present disclosure, the chip and/or antenna comprises or consists of the sensing circuit.

### Reagents

The porous phase of the lateral flow device may be configured in a variety of ways to accommodate the need for targeting different analytes in liquid samples. As such, the binding molecule may also be varied accordingly.

Thus, in one embodiment of the present disclosure, the at least one binding molecule is an antibody.

Aptamers have unique affinity toward target molecules, and are preferred over antibodies due to features such as easy production, high stability, reproduction and versatility of applications as well as simple labelling, amplification and selection processes.

Thus, in one embodiment of the present disclosure, the at least one binding molecule is an aptamer.

Thus, in one embodiment of the present disclosure, the at least one binding molecule is an affimer.

Thus, in one embodiment of the present disclosure, the at least one binding molecule is an RNA molecule.

Thus, in one embodiment of the present disclosure, the at least one binding molecule is a DNA molecule.

Thus, in one embodiment of the present disclosure, the at least one binding molecule is an organic polymer.

Thus, in one embodiment of the present disclosure, the at least one binding molecule is any one of the binding molecules described herein, or a fragment thereof.

In a preferred embodiment of the present disclosure at least one of the binding molecule and the capture molecule is specific for a biological marker.

Further, in a specific embodiment of the present disclosure, the sample is a bodily sample.

Likewise, the read-out from the analytical device to the external reader unit is based on the sensing circuit's ability to recognize certain analytes in liquid samples. This is achieved by implementing capture molecules to the detection zone which match the binding molecules described herein. As such, the capture molecule may also be varied accordingly.

Thus, in one embodiment of the present disclosure, the at least one capture molecule is the analyte.

Thus, in one embodiment of the present disclosure, the at least one capture molecule is a binding equivalent of the analyte.

Thus, in one embodiment of the present disclosure, the at least one capture molecule is an antibody.

Thus, in one embodiment of the present disclosure, the at least one capture molecule is an aptamer.

Thus, in one embodiment of the present disclosure, the at least one capture molecule is an affimer.

Thus, in one embodiment of the present disclosure, the at least one capture molecule is an RNA molecule.

Thus, in one embodiment of the present disclosure, the at least one capture molecule is a DNA molecule.

Thus, in one embodiment of the present disclosure, the at least one capture molecule is an organic polymer.

Thus, in one embodiment of the present disclosure, the at least one capture molecule is any one of the capture molecules described herein, or a fragment thereof.

The read-out signal from the sensing circuit may be dependent on the amount of metal reporter present in the detection zone.

Thus, in one embodiment of the present disclosure, at least one of the binding molecules or the capture molecules comprise the metal reporter.

In one embodiment of the present disclosure, the metal reporter is a metal colloid. A person skilled in the art will appreciate that any colloidal metal may be used in the present disclosure. Colloidal metal includes any water-insoluble metal particle or metallic compound dispersed in liquid water, a hydrosol or a metal sol. Preferred metals include gold, silver and platinum.

### Analytical Device Format

In specific embodiments of the present disclosure the application zone comprises the one or more detection zones. As such, the application zone may overlap, completely or partially with the detection zone(s). The detection zones may for example be covered by a membrane for filtering of the liquid sample, e.g. filtering of particulate matter. Further, the application zone may comprise a vial for receiving the liquid sample, and said vial may comprise at least a part of the sensing circuit, i.e. at least the detection zones.

### Lateral Flow Assay (LFA)

In specific embodiments of the present disclosure, the analytical device may comprise or consist of a lateral flow assay. The analytical device may thereby comprise a porous phase (e.g. a membrane layer) configured for wicking or the liquid sample, such as from the application zone to the at least one detection zone. The device may be divided into multiple zones. For example the device may comprise an application zone and a detection zone. The detection zone(s) may be provided in a membrane. The device may further comprise an absorbent pad for improved wicking of fluid through the porous phase. It is a further preference that the device comprises at least one sensor and transmitter zone.

Thus, in one embodiment of the present disclosure, the application zone of said analytical device comprises, in the dry unused state, at least one binding molecule, and/or wherein said sample has been premixed with the at least one binding molecule.

Application zone refers to the part of the analytical device that is configured for receiving the liquid sample, such as, but not limited to, a sample of blood, urine or interstitial fluid. The application zone may comprise binding molecules, for example in a separate binding molecule zone, e.g. a conjugate pad. Upon application of the liquid sample, the analyte(s) may interact with the binding molecule(s) and form complexes by specific binding. The application zone may contain several specific molecules, each capable of specific binding to a different analyte.

In specific embodiments of the present disclosure, the analytical device may comprise an application zone that is separated from the one or more detection zones, for example wherein the analytical device comprises a porous phase for wicking of the liquid sample from the application zone to the one or more detection zones. Alternatively or additionally, the application zone comprises the detection zone, e.g. the application zone overlaps, completely or partially with the detection zone. The detection zone may for example be covered by a membrane for filtering of the liquid sample, e.g. filtering of particulate matter.

In one embodiment of the present disclosure, said analytical device comprises an absorbent pad. An absorbent pad allows for controlled release of sample and conjugate onto the analytical device and to filter undesired components. The absorbent pad may be in any type of material that absorbs water, such as cellulose fiber or woven meshes.

In one embodiment of the present disclosure, said analytical device comprises a sample pad, comprising the application zone, such as in cellulose acetate and/or glass fiber.

In one embodiment of the present disclosure, said analytical device comprises a conjugate release pad.

In one embodiment of the present disclosure, said analytical device comprises a membrane.

In one embodiment of the present disclosure, said membrane is nitrocellulose.

In one embodiment of the present disclosure, the absorbent pad, the sample pad, the conjugate release pad and the membrane of said analytical device are fluidly connected.

In one embodiment of the present disclosure, said analytical device comprises a backing layer in an inert material. Inert materials are materials which have minimal or almost no chemical or biological activity when present in the environment.

In one embodiment of the present disclosure, said analytical device comprises a cassette.

In one embodiment of the present disclosure, said analytical device is configured for being used as a dipstick. Consequently, the analytical device may be configured such that the sample may be applied to the application zone, by dipping the analytical device in the liquid sample.

In a further embodiment of the present disclosure, the analytical device is reusable. The analytical device may for example be cleaned by any suitable cleaning agent, such as phosphate-buffered saline. Cleaning may be performed by immersing the analytical device in the cleaning agent and or by rinsing of the analytical device in the cleaning agent. Preferably, the analytical device is configured such that the main part of the liquid sample is removed by said cleaning. Alternatively or additionally to cleaning by liquid cleaning agent, the cleaning agent may be in gaseous phase, such as water vapor. Cleaning may for example comprise an autoclaving process, wherein the analytical device is sterilized, typically by subjecting the analytical device to water vapor at elevated temperatures and pressures.

In another embodiment of the present disclosure, the analytical device is intended for single-use only. For specific applications, single-use devices may be preferred. The analytical device may for example be provided sterilized in a sealed environment. Following use, at least parts of the analytical device may be disposed of. Parts of a single-use analytical device that may be intended to be disposed of following use typically include parts that have, or suspected of having, been subjected to the liquid sample and/or the ambient environment.

### Measurement Results

The analytical device of the present disclosure is configured in such a way that it is able to provide quantitative measurements of analytes in biological liquid samples.

Thus, in one embodiment of the present disclosure, said analytical device is configured such that an increase in the analyte concentration results in an increase in the amount of the metal reporter at the at least one detection zone.

In one embodiment of the present disclosure, said analytical device is configured such that an increase in the analyte concentration results in a decrease in the amount of the metal reporter at the at least one detection zone.

In one embodiment of the present disclosure, said analytical device is configured for operation in sandwich assay format and/or competitive assay format. A person skilled in the art will appreciate that competitive assays generally are used for smaller compounds, but is also preferable for compounds with multiple epitopes.

In one embodiment of the present disclosure, said analytical device comprises multiple detection zones, for measuring of the concentration of multiple analytes.

In one embodiment of the present disclosure, said analytical device comprises a control zone configured for, following a measurement, providing an indication that the analytical device has operated correctly.

In one embodiment of the present disclosure, said analytical device further comprises a vessel with a binding solution comprising the binding molecules. The binding molecules may be configured to bind to the analyte, typically after the analyte has bound to the capture molecule at the at least one detection zone.

### System

A further aspect of the present disclosure relates to an analytical system for measuring the concentration of at least one analyte in a liquid sample, said system comprising:
a. an analytical device, such as disclosed elsewhere herein;
b. an external reader unit configured for receiving a return signal, said return signal representative of the analyte concentration and/or changes in the electrical property of the at least one detection zone.

In one embodiment the analytical device comprises a porous phase for wicking of liquid between the application zone and the detection zone, e.g. the analytical device may be a lateral flow device.

In an embodiment of the present disclosure the system may comprise an external reader unit configured for generating the interrogating signal for powering the electrical device of the analytical device, the external reader unit being further configured for receiving and interpreting a radio-wave based return signal from the electrical device, said return signal representative of the analyte concentration and/or changes in the electrical property of the at least one detection zone.

### External Reader Unit

The present disclosure provides a way of reading the result from the analytical device on an external reader unit.

In one embodiment of the present disclosure, the external reader unit of said system is a portable device, such as a smartphone or a smartwatch. The external reader unit may consequently be wearable and/or hand-held.

In one embodiment of the present disclosure, the external reader unit of said system is configured for communication with the analytical device, such as the electrical device, by near-field communication (NFC), Bluetooth, Bluetooth low energy (BLE) and/or Wi-Fi.

### Method

Analytical devices sometimes have a binary outcome, for example most lateral-flow tests. An analytical device with a capability to quantitatively measure the concentration of an analyte in a liquid sample and configured to transmit a return signal representative of the analyte concentration an external reader unit, for use in home-testing or point-of-care testing, would provide significant benefits to the user.

In a further aspect, the present disclosure relates to a method for measuring the concentration of an analyte in a liquid sample, said method comprising the steps of:
a. providing an analytical system, such as disclosed elsewhere herein;
b. applying a liquid sample comprising analytes to the application zone;
c. generating, by the electrical device, a radio-wave based return signal representative of the analyte concentration;
d. receiving, by the external reader unit, the return signal; and
e. optionally, interpreting said return signal;
f. thereby measuring the concentration of the analyte in the liquid sample.

In an embodiment of the present disclosure, the method may comprise a step of: allowing migration of the analytes from the application zone to the at least one detection zone.

In an embodiment of the present disclosure, the method may comprise a step of: generating, by an external reader unit, a radio-wave based interrogating signal. Preferably said step is perform before the step of generating a return signal. The method may further comprise a step of: receiving, by the electrical device, the interrogating signal, such that a current flows through the sensing circuit. Preferably said step is perform before the step of generating a return signal.

In one embodiment of the present disclosure, the sample of said method has been pretreated, such as mixed with a binding solution comprising binding molecules.

In a specific embodiment of the present disclosure, the steps are performed in the following order:
providing an analytical system, such as disclosed elsewhere herein;
applying a liquid sample comprising analytes to the application zone;
generating, by the electrical device, a radio-wave based return signal representative of the analyte concentration;
receiving, by the external reader unit, the return signal; and
interpreting said return signal;
a. thereby measuring the concentration of the analyte in the liquid sample.

In an embodiment of the present disclosure, the method further comprises a step, between steps 2. and 4., such as between 2. and 3. or between 3. and 4.: allowing migration of the analytes from the application zone to the at least one detection zone. The analytical device may thereby comprise a porous phase arranged such that at least a part of the liquid sample migrates, upon application, from the application zone to the at least one detection zone.

In an embodiment of the present disclosure, the method further comprises a step, between step 2. and 4.: generating, by the external reader unit, a radio-wave based interrogating signal.

In an embodiment of the present disclosure, the method further comprises a step, before step 1: processing of the sample, wherein said processing may comprise of consist of stabilizing the sample.

### DETAILED DESCRIPTION OF THE DRAWINGS

The invention will in the following be described in greater detail with reference to the accompanying drawings. The drawings are exemplary and are intended to illustrate some of the features of the presently disclosed analytical device, analytical system, and method of measuring the concentration of an analyte in a liquid sample, and are not to be construed as limiting to the presently disclosed invention.

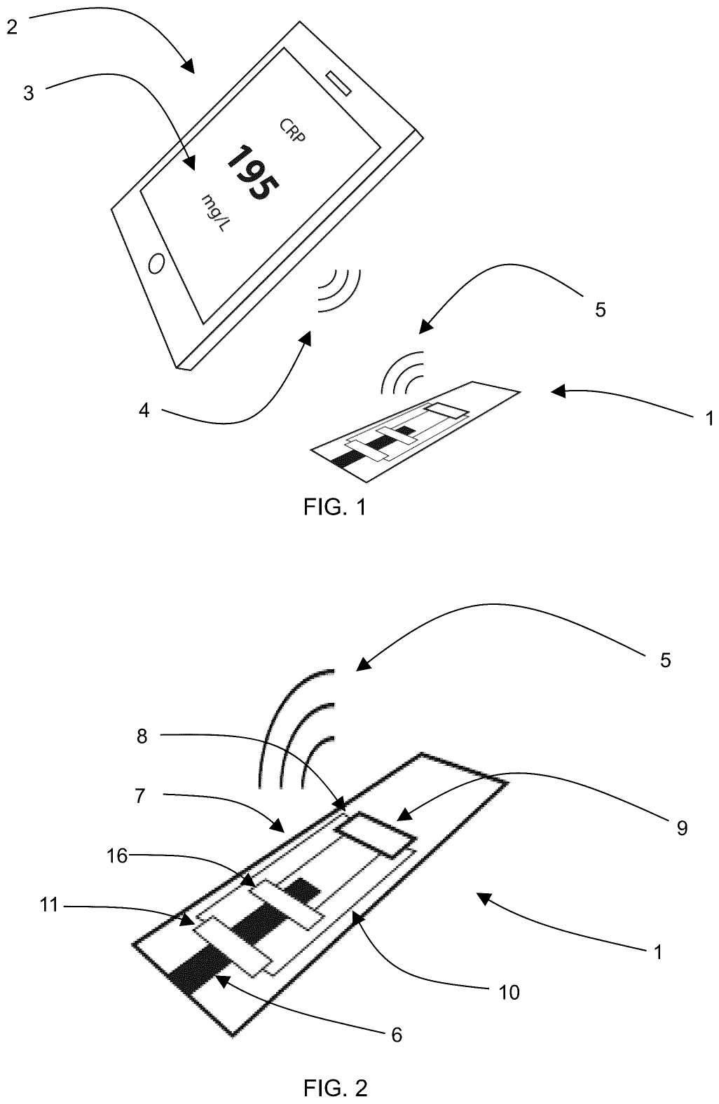

FIG. 1 shows a schematic illustration of a preferred embodiment of the present disclosure. Herein the analytical device (1) can be seen to be configured for communicating with an external reader unit (2). The external reader unit may be a portable device, for example a hand-held smartphone. Preferably the external reader unit comprises a display for providing a user with for example connection status between the external reader unit and the analytical device and measurement information (3). A smartphone may for example comprise an app, that is configured to guide a user through a measurement session, and following said session, displaying a measurement result to said user, for example the concentration of one or more analytes in one or more liquid samples. In a specific embodiment of the present disclosure, the external reader unit and the analytical device is configured for communicating over any suitable wireless connection standard. For example Bluetooth, ZigBee, RFID, WiFi, NFC, or ultrawide band. It is a preference that the analytical device is configured such that it may receive an interrogation signal (4) from the external reader unit. Said interrogation signal may be in a suitable wireless standard format. It is a further preference that the analytical device is at least in part powered by said interrogation signal. Preferably the analytical device is configured to transmit, in response to the interrogation signal, a return signal (5) that is representative of the analyte concentration in the liquid sample.

FIG. 2 shows a schematic illustration of selected features of an exemplary analytical device (1) of the present disclosure. Herein, it can be seen that the analytical device is configured to transmit a return signal (5). It is a preference that the return signal is sent out in response to receiving an interrogation signal, from an external reader unit. The return signal is typically representative of the one or more analyte concentration of the one or more samples. However, it should be noted that the return signal may comprise further information, in addition to information representative of said analyte(s) concentration. For example the return signal may comprise device information or test information. Device information may be any information related to the specific analytical device any may for example comprise an identification code of the analytical device, said identification code may be provided as a number and may be unique for each analytical device. The test information is preferably any information related to what and how the analytical device is configured to measure. For example, what type and what concentration of binding molecules and capture molecules said device has, or what sample volume should be added to the device. In short, said test information is any information that may help a user understand how to use said device or provide a user with specific information about the analytical device.

In a preferred embodiment, with reference to FIG. 2, the analytical device comprises an application zone (6) for receiving samples or any further liquids, such as a running buffer. Typically, but not necessarily, the application zone is located near an edge of the analytical device, such as a proximal end of the analytical device. This may enable dipping of the analytical device in a liquid (e.g. a liquid sample) easier. However, liquids may be added by other means, for example pipetting. Following application of said liquids to the analytical device, the analyte migrates towards the detection zones. Typically, the migration of the analytes is enabled by capillary action through the porous phase of the analytical device. Said device may comprise an absorbent pad (9) for prolonged wicking of the sample. The analytical device preferably comprises an electrical device (7). The electrical device may comprise an antenna (8), a chip (9), and a sensing circuit (10). It is a preference that the sensing circuit comprises one or more detection zones (11). Said detection zones may be provided towards a distal end of the analytical device, with respect to the application zone, and may comprise an immobilized capture molecule configured for specific interaction with the analyte and/or the binding molecule. Preferably said capture molecules and binding molecules are configured such, together with the analyte, that said interaction results in a change in at least one electrical property of the at least one detection zone by modifying the amount of a metal reporter of said detection zone. Each detection zone may comprise a single type of capture molecule, and consequently, each detection zone may target a specific analyte of the liquid sample. It is a further preference that the electrical device is configured such that, upon receiving an interrogation signal from the external reader unit, a current flows through the sensing circuit and is modified into a return signal representative of the analyte concentration, by the change in the at least one electrical property of the detection zone, said electrical device further being configured for transmitting said return signal to the external reader unit. As can be seen in FIG. 2, this exemplary analytical device comprises a sensing circuit comprising one detection zone (11) and one reference zone (16), each being electrically connected to the chip. Typically, the reference zone is similar to a detection zone, but does not comprise any capture molecules. By the use of a reference zone, background noise may be recorded and used for processing of the signal obtained from the detection zone(s). Although not shown, the analytical device may further comprise a control zone that is configured to indicate if the analytical device has operated correctly, i.e. similar to a control zone/control line of a conventional analytical device. The control zone of the present disclosure may for example comprise control molecules directed at immobilization of the binding molecules, wherein said immobilization leads to a change in at least one electrical property of said control zone. If the detection zone(s), following interaction between the liquid sample/analyte and the capture molecules, has a modified amount of the metal reporter, a current through said detection zone may be modified based on the change in the at least one electrical property. The modified current is used to form the return signal, and said return signal may thereby be representative of the one or more analyte concentrations in the sample. The return signal is transmitted by an antenna (8). The antenna may be an integral part of the chip, a separate antenna that is electrically connected to the chip, and/or be provided as at least a part of the sensing circuit.

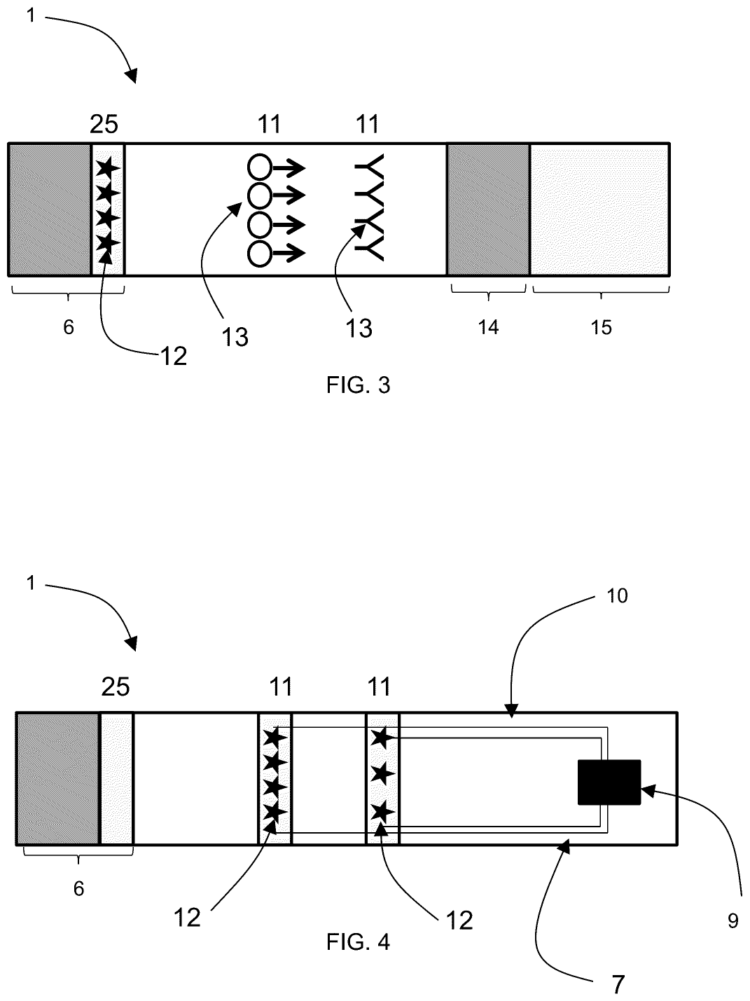

FIG. 3 is a schematic illustration of a further analytical device (1) of the present disclosure. This exemplary analytical device comprises an application zone (6) near a proximal end of said device. The application zone being configured for receiving a sample. Once the sample has been provided to the analytical device, the device is configured such that said sample will migrate towards a distal end of said device. In this specific example the application zone comprises binding molecules (12), that in the dry-unused state is located in a binding molecule zone (25). The application of the sample leads to an interaction between the sample and the binding molecules and may lead to the formation of a complex comprising said binding molecules and the analyte(s). The binding molecules may comprise or consist of any of an antibody, an aptamer, an affimer, an RNA molecule, a DNA molecule, an organic polymer, or a fragment thereof, or a combination thereof. It should be noted that the binding molecules may additionally or alternatively be provided separate from the analytical device. In such an instance, the liquid sample may be mixed with the binding molecules before applying the liquid sample to the analytical device. The sample preferably further interacts with the capture molecules (13) of each detection zone while the sample migrates towards the absorbent pad (14). The analytical device may comprise a sensor and transmitter zone (15) wherein the main part of the electrical device is located, typically at least the chip. However, parts of the electrical device may be located outside said sensor and transmitter zone, for example at least part of the detection zone(s), the sensing circuit(s) and the antenna.

FIG. 4 shows a schematic illustration of an exemplary analytical device (1), focusing on the electronic parts thereof. Specifically, this figure illustrates an analytical device following addition of a liquid sample to the application zone (6) and wherein the liquid sample has interacted with the binding molecules (12) that initially were located in the binding molecule zone (25). The analytes and the binding molecules have thereafter formed complexes, wherein a specific type of binding molecules may form complexes with only a specific type of analytes, and/or the binding molecules may form complexes with all types of analytes. The formed complexes have migrated towards an absorbent pad and interacted with the capture molecules of the detection zones (11). Depending on the type of analyte and/or binding molecules, the analyte may be immobilized at a specific one of the detection zones. In the shown illustrative example, the analytes (not shown) together with the binding molecules (12) have been immobilized at the detection zones (13). Herein, the binding molecules may comprise a metal reporter, and thereby the amount of the binding molecules present at the detection zone may be equal to the amount of the metal reporter. Consequently, upon immobilization of the analyte-binding molecule complex on each detection zone, the at least one electrical property of said detection zone is changed. This is one type of configuration that may lead to a change in the at least one electrical property of the at least one detection zone. As disclosed elsewhere herein, several different configurations are possible that will lead to the same result and all are within the scope of the present disclosure. The analytical device further comprises an electrical device (7), herein exemplified as comprising a chip (9) and a sensing circuit, wherein the sensing circuit comprises the detection zones. The electrical device further comprises an antenna, which may be provided as at least a part of the sensing circuit and/or the chip. In other embodiments it may however be provided as a separate unit, preferably electrically connected to the chip. The electrical device may preferably be configured such that, upon receiving an interrogation signal from an external reader unit, a current flows through the sensing circuit and is modified into a return signal that is representative of the analyte concentration, by the change in the at least one electrical property of the detection zone. The electrical device may further be configured for transmitting said return signal to the external reader unit. The modification of the interrogation signal into the return signal may be carried out in multiple steps, wherein the change in the at least one electrical property of the detection zones may be one step. However, additional steps may be carried out by other parts of the sensing circuit, such as a sensor unit for controlling the sensing circuit, the chip and/or the antenna. Furthermore, the external reader unit may comprise programming instructions that, when executed, are able to process the received return signal and derive measurement data, such as measurement values, test information and device information.

FIG. 5A shows a schematic illustration of a further exemplary analytical device (1) comprising a porous phase having an application zone (6) at a proximal end of said device and an absorbent pad (14) towards a distal end of said device, with respect to the application zone. The analytical device further comprises an electrical device (7) comprising an antenna (8), a chip (9) and a sensing circuit (10). In this specific example, the antenna is provided as a separate unit, that is in electrical connection with the chip. This is often a standard configuration in radio-wave based devices, such as RFID tags. The chip may thereby be configured for radio-wave based communication with an external reader unit, using the antenna. The sensing circuit is in this example provided as a separate circuit from the other components of the electrical device, although electrically connected to the chip. The sensing circuit comprising two detection zones (11), and a sensing unit (26). The sensing unit is preferably in electrical connection with the chip. In this configuration the sensing unit may, typically following input from the chip, control and/or measure at least one electrical property of the sensing circuit, wherein the electrical property may change depending on the amount of a metal reporter present at the detection zone(s). The sensing unit may further provide obtained and/or measured information to the chip, in unprocessed or processed format. Unprocessed format could for example be measurement values of one or more electrical properties while a processed format could be concentration values of the one or more target analytes in the sample. The return signal is formed by the sensing unit, the chip, and/or the antenna, and is preferably based on the change in the at least one electrical property, due to the modification of the amount of the one or more metal reporters of the detection zone(s).

FIG. 5B shows a schematic illustration of a further exemplary analytical device (1) comprising a porous phase having an application zone (6) at a proximal end of said device and an absorbent pad (14) towards a distal end of said device, with respect to the application zone. The analytical device further comprises an electrical device (7) that comprises an antenna (8), a chip (9) and a sensing circuit (10). In this specific example, the sensing circuit, in addition to comprising the detection zone(s) also comprises the antenna. By having such a configuration, the current that flows through the sensing circuit, typically upon receiving an interrogation signal from an external reader unit, may be modified into the return signal substantially simultaneously, or simultaneously as transmitting said signal. For example, a part of the antenna may be provided in the porous phase, and wherein said part of the antenna may comprise immobilized capture molecules. It should be noted that although not shown the electrical device may comprise at least one antenna for receiving the interrogation signal and at least one antenna for transmitting the return signal. In such an instance, at least a part of the at least one antenna may comprise the capture molecules. Irrespective of the components of the electrical device and how they communicate with each other, it is a preference that the electrical device is configured such that depending on the concentration of the target analyte(s) in the sample, the amount of the metal reporter at each detection zone is modified. As a result at least one electrical property of the detection zone(s) is changed, and the transmitted return signal(s may thereby comprise information representative of the concentration of the target analyte(s) in the sample, in addition to further information such as test information and device information as mentioned elsewhere herein.

FIG. 5C shows a schematic illustration of a further exemplary analytical device (1) comprising a porous phase having an application zone (6) at a proximal end of said device and an absorbent pad (14) towards a distal end of said device, with respect to the application zone. The analytical device further comprises an electrical device (7) comprising multiple antennas (8), multiple chips (9) and multiple sensing circuits (10). In this specific example, each set of antenna, chip and sensing circuit may provide a separate return signal comprising different information. In this specific example, one of the sensing circuits (left) comprises a detection zone (11) wherein the amount of a metal report may be modified depending on the concentration of the analyte present in the sample. The other sensing circuit (right) instead comprises a reference zone (16) that may be configured to measure the background, such as a background signal. For example the reference zone may be substantially identical to a detection zone, but may lack the capture molecules, such as any capturing molecules. The return signal for the sensing circuit comprising a reference zone may, following application of the sample, be subjected to a change in at least one electrical property. Thereby, the return signal from this part of the electrical device may comprise information about the background level of the signal. A person skilled in the art is aware that the above stated example may be carried out in many different configurations, for example the electrical device may comprise one or more chips, one or more antennas, and one or more sensing circuits. However, it is a preference that the electrical device comprises at least one detection zone and, in specific embodiments, a reference zone for measuring the background level.

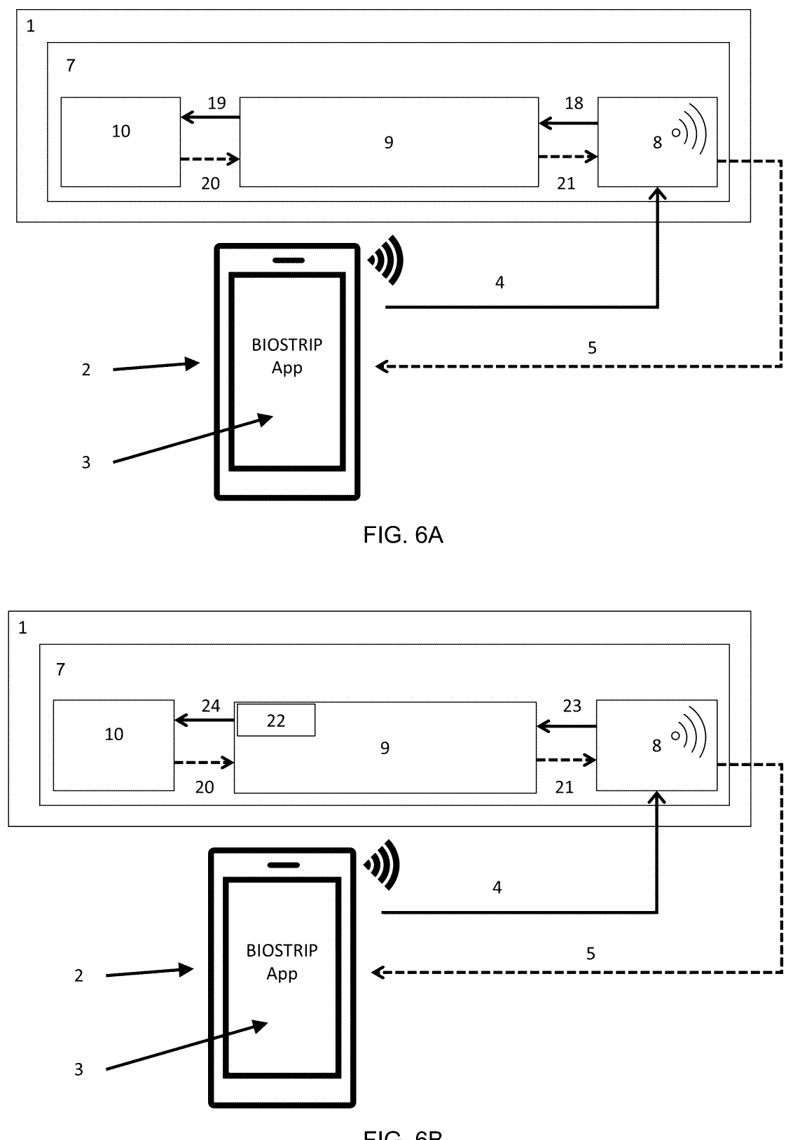

FIG. 6A shows a flow chart for the communication between the external reader unit, the analytical device, and the components of the analytical device, according to a specific embodiment of the present disclosure. In this example, the electrical device (7) of the analytical device (1) is powered by the interrogation signal (4) transmitted from an external reader unit (2). A user may for example, through a user interface (3) instruct the external reader unit to transmit said interrogation signal to the electrical device. The analytical device is configured to receive said interrogation signal by an antenna (8). The electrical device is preferably configured such that the received interrogation signal is transmitted to the sensing circuit (10), potentially, as shown, through the chip (9) first. As previously discussed, a modified amount of a metal reporter, and consequently a change in at least one electrical property, of the detection zone, may be used in the formation of a return signal. It should however be noted that the return signal may be processed by at least the chip and/or the antenna before being transmitted by the antenna. In the given example, the sensing circuit produces an output signal that could be referred to as an unprocessed return signal (20). Further, this signal is fed to the chip which may modify the signal into a processed return signal (21). The processed return signal may comprise additional information or may have had measurement data processed, such as having background omitted or having measurement data converted to analyte concentration. The antenna thereafter transmits the return signal (in this case the processed return signal) (5). The transmitted return signal may thereafter be received by the external reader unit, that may display or record values of one or more concentrations of the analyte(s) in the liquid sample.

FIG. 6B shows a flow chart for the communication between the external reader unit, the analytical device, and the components of the analytical device, according to a specific embodiment of the present disclosure. In this example, the electrical device (7) of the analytical device (1) is configured to receive an interrogation signal (4) from an external reader unit (2). The analytical device is configured to receive said interrogation signal by the antenna (8). The chip may further transmit the received interrogation signal, or a signal derived therefrom, to the chip (9). The chip may comprise, or be connected to, such as electrically connected to, a battery (22). In this embodiment of the present disclosure, it may be a preference that the battery may power, at least partly, the electrical device. For example, the battery may provide power to the sensing circuit (10) and/or the chip. The battery may provide power to the sensing circuit (24), and depending on the concentration of the analyte the power is used to generate a return signal (20). Said signal may be provided to the chip and processed before being provided to the antenna (21). As discussed elsewhere herein, several different configurations of the electrical device are possible and within the scope of the present disclosure.

### Items

1. An analytical device for measuring the concentration of an analyte in a liquid sample, said analytical device comprising:
   a porous phase comprising an application zone for receiving the sample, towards a proximal end of said porous phase, and wherein the porous phase is configured such that, following receiving said sample, the analyte and optionally at least one binding molecule migrate towards a distal end of said porous phase;
   an electrical device comprising:
      an antenna;
      a chip configured for radio-wave based communication with an external reader unit, using the antenna;
      a sensing circuit comprising at least one detection zone, provided towards a distal end of said porous phase, having an immobilized capture molecule configured for specific interaction with the analyte and/or the binding molecule such that said interaction results in a change in at least one electrical property of the at least one detection zone by modifying the amount of a metal reporter of said detection zone;
wherein the electrical device is configured such that, upon receiving an interrogation signal from the external reader unit, a current flows through the sensing circuit and is modified into a return signal representative of the analyte concentration, by the change in the at least one electrical property of the detection zone, said electrical device further being configured for transmitting said return signal to the external reader unit.
2. The analytical device according to an one of the preceding items, wherein the analytical device is a lateral flow device.
3. The analytical device according to an one of the preceding items, wherein the sensing circuit is provided as a circuit separate from the antenna and the chip, and wherein the sensing circuit is in electrical contact with the antenna and/or the chip.
4. The analytical device according to any one of the preceding items, wherein the sensing circuit comprises at least a part of the chip.
5. The analytical device according to any one of the preceding items, wherein the sensing circuit comprises at least a part of the antenna.
6. The analytical device according to any one of the preceding items, wherein the sensing circuit comprises at least two detection zones, such as for measuring multiple analyte concentrations.
7. The analytical device according to any one of the preceding items, wherein the sensing circuit comprises a reference zone configured for measuring a baseline of a measured electrical property, such as being located towards the distal end of said porous phase and wherein said reference zone does not comprise immobilized capture molecules.
8. The analytical device according to any one of the preceding items, wherein the at least one detection zone and/or reference zone comprises a conductive film layer covering a part of the porous phase.
9. The analytical device according to any one of the preceding items, wherein the at least one electrical property is selected among the group of: electrical impedance, such as the complex impedance, electrical resistance, electrical conductance, dielectric constant, frequency tuning, activation power, received signal strength indicator, or a combination thereof.
10. The analytical device according to any one of the preceding items, wherein the antenna is configured for generating a radio-wave based return signal, said return signal representative of the measured electrical property and/or the analyte concentration.
11. The analytical device according to any one of the preceding items, wherein the electrical device comprises a processing unit configured for processing data associated with the measurement, such as for computing the analyte concentration.
12. The analytical device according to any one of the preceding items, wherein the interrogating signal is configured for powering the electrical device.
13. The analytical device according to any one of the preceding items, wherein the electrical device is configured for being powered solely by the interrogating signal.
14. The analytical device according to any one of the preceding items, wherein the electrical device consists or comprises an RFID tag, an NFC tag, a Wi-Fi tag, an ultra-wide band tag, a Bluetooth tag, and/or a ZigBee tag.
15. The analytical device according to any one of the preceding items, wherein the antenna is omnidirectional or hemispherical such that it is configured to receive the interrogating signal when the external reader unit is out of sight.
16. The analytical device according to any one of the preceding items, wherein the at least one binding molecule is selected from the list of an antibody, an aptamer, an affimer, an RNA molecule, a DNA molecule, an organic polymer, or a fragment thereof.
17. The analytical device according to any one of the preceding items, wherein the at least one capture molecule is selected from the list of the analyte, a binding equivalent of the analyte, an antibody, an aptamer, an affimer, an RNA molecule, a DNA molecule, an organic polymer, or a fragment thereof.
18. The analytical device according to any one of the preceding items, wherein at least one of the binding molecules or the capture molecules comprise the metal reporter.
19. The analytical device according to any one of the preceding items, wherein the metal reporter is a metal colloid.
20. The analytical device according to any one of the preceding items, wherein the application zone comprises, in the dry unused state, at least one binding molecule, and/or wherein said sample has been premixed with the at least one binding molecule.
21. The analytical device according to any one of the preceding items, wherein said device comprises an absorbent pad.
22. The analytical device according to any one of the preceding items, wherein said device comprises a sample pad, comprising the application zone, such as in cellulose acetate and/or glass fiber.
23. The analytical device according to any one of the preceding items, wherein said device comprises a conjugate release pad.
24. The analytical device according to any one of the preceding items, wherein said device comprises a membrane, such as in nitrocellulose.
25. The analytical device according to any one of the preceding items, wherein the absorbent pad, the sample pad, the conjugate release pad and the membrane are fluidly connected.
26. The analytical device according to any one of the preceding items, wherein said device comprises a backing layer in an inert material.
27. The analytical device according to any one of the preceding items, wherein the analytical device comprises a cassette, and/or wherein the analytical device is configured for being used as a dipstick.
28. The analytical device according to any one of the preceding items, wherein the analytical device is configured such that an increase in the analyte concentration results in an increase in the amount of the metal reporter at the at least one detection zone.
29. The analytical device according to any one of the preceding items, wherein the analytical device is configured such that an increase in the analyte concentration results in a decrease in the amount of the metal reporter at the at least one detection zone.
30. The analytical device according to any one of the preceding items, wherein the analytical device is configured for operation in sandwich assay format and/or competitive assay format.
31. The analytical device according to any one of the preceding items, wherein said device comprises multiple detection zones, for measuring of the concentration of multiple analytes.
32. The analytical device according to any one of the preceding items, wherein the analytical device comprises a control zone configured for providing an indication, following a measurement, that the analytical device has operated correctly.
33. The analytical device according to any one of the preceding items, further comprising a vessel with a binding solution comprising the binding molecules.
34. An analytical system for measuring the concentration of at least one analyte in a liquid sample, said system comprising:
   the analytical device according to any one of the items 1-33;
   an external reader unit configured for generating the interrogating signal for powering the electrical device of the analytical device, the external reader unit being further configured for receiving and interpreting a radio-wave based return signal from the electrical device, said return signal representative of the analyte concentration and/or changes in the electrical property of the at least one detection zone.
35. The system according to item 34, wherein the external reader unit is a portable device, such as a smartphone.
36. The system according to any one of items 34-35, wherein the external reader unit is configured for near-field communication (NFC) with the electrical device.
37. A method for measuring the concentration of an analyte in a liquid sample, said method comprising the steps of:
   providing the analytical system according to any one of items 34-36;
   applying a liquid sample comprising analytes to the application zone;
   allowing migration of the analytes from the application zone to the at least one detection zone;
   generating, by an external reader unit, a radio-wave based interrogating signal;
   receiving, by the electrical device, the interrogating signal, such that a current flows through the sensing circuit; and
   generating, by the electrical device, a radio-wave based return signal representative of the analyte concentration;
   receiving, by the external reader unit, the return signal; and
   interpreting said return signal;
   thereby measuring the concentration of the analyte in the liquid sample.
38. The method according to item 37, wherein the sample has been pretreated, such as mixed with a binding solution comprising binding molecules.

1-34. (canceled)

35. An analytical device for continuous and specific measurements of the concentration of an analyte in a liquid sample, said device comprising:
an application zone for receiving the sample; wherein said application zone comprises a binding molecule selected from the group including an antibody, an aptamer and an affimer, the binding molecule having a metal reporter and configured for specific binding of the analyte to form an analyte-binding molecule complex:
an electrical device comprising:
a chip and an antenna configured for continuous bidirectional radio-wave communication with an external reader unit;
a power source;
a sensing circuit comprising at least one detection zone, having an immobilized capture molecule selected from the group including an antibody, an aptamer and an affimer, the capture molecule configured for specific binding of the analyte and wherein binding of the analyte-binding molecule complex by the capture molecule results in a change in at least one electrical property of said detection zone by modification of an amount of the metal reporter of the detection zone;
wherein the electrical device is configured such that,
upon receiving, by the chip and antenna, a radio-wave based interrogation signal from the external reader unit, a current flows through the sensing circuit; or
the chip initiates a flow of a current through the sensing circuit;
and wherein the electrical device is further configured to, continuously and uninterruptedly, modify, based on the change in the at least one electrical property of the detection zone, the current into a return signal and transmit said return signal to the external reader unit by the chip and the antenna, and wherein the return signal is representative of the analyte concentration.

36. The analytical device according to claim 35, wherein the device is configured to transmit the return signal to the external reader unit by near-field communication (NFC), Wi-Fi, ultra-wide band (UWB), Bluetooth, Bluetooth low energy (BLE), Low Power Wide Area Network (LPWAN) and/or ZigBee.

37. The analytical device according to claim 35, wherein the application zone comprises the sensing circuit and/or the at least one detection zone.

38. The analytical device according to claim 35, wherein the at least one electrical property is selected among the group of electrical impedance, such as the complex impedance, electrical resistance, electrical conductance, dielectric constant and frequency tuning, or a combination thereof.

39. The analytical device according to claim 35, wherein the electrical device is configured for being powered by the wireless interrogating signal, an internal power source, such as a battery, or a combination thereof.

40. The analytical device according to claim 35, wherein the sensing circuit comprises at least two detection zones adapted to measure the concentration of at least two different analytes.

41. The analytical device according to claim 40, wherein the electrical device is adapted to generate a single return signal representative of the concentrations of the at least two analytes.

42. The analytical device according to claim 35, wherein the sensing circuit comprises a reference zone, such as one reference zone for each detection zone, wherein the one or more reference zones are configured for measuring a reference value, such as a baseline, of a measured electrical property and/or an analyte concentration.

43. The analytical device according to claim 35, wherein the device comprises a porous phase, and wherein the porous phase is configured such that, following receiving said sample at the application zone, the analyte and optionally at least one binding molecule migrate to the detection zone, located towards a distal end of said porous phase.

44. The analytical device according to claim 35, wherein the antenna and the chip are not part of the sensing circuit, and wherein the sensing circuit is in electrical contact with the antenna and/or the chip.

45. The analytical device according to claim 35, wherein the chip and/or antenna comprises or consists of the sensing circuit.

46. The analytical device according to claim 35, wherein the electrical device is configured such that the flow of current is generated into the return signal by the change in the at least one electrical property.

47. An analytical system for continuous measurements of the concentration of at least one analyte in a liquid sample, said system comprising:
an analytical device according to claim 35; and
an external reader unit configured for receiving a return signal, said return signal representative of the analyte concentration and/or changes in the electrical property of at least one detection zone.

48. The analytical system according to claim 47, wherein the external reader unit is configured for transmitting the radio-wave based interrogating signal for powering the electrical device of the analytical device.

49. The analytical system according to claim 47, wherein the external reader unit is configured for receiving and interpreting a radio-wave based return signal from the electrical device.

50. A method for measuring the concentration of an analyte in a liquid sample, said method comprising the steps of:
a. providing the analytical system according to claim 47;
b. applying a liquid sample comprising analytes to the application zone:
c. generating, by the electrical device, a radio-wave based return signal representative of the analyte concentration; and
d. receiving, by the external reader unit, the return signal.

51. The method according to claim 50, further comprising step e. interpreting the return signal for deriving the analyte concentration.

52. The method according to claim 50, further comprising, between step b) and c): allowing migration of the analytes from the application zone to the at least one detection zone.

53. The method according to claim 50, further comprising step of generating, by the external reader unit, a radio-wave based interrogating signal.

54. The method according to claim 50, further comprising, before step a), processing of the sample, such as stabilizing the sample.
